(19) United States
(12) Patent Application Publication
(10) Pub. No.: US 2012/0094008 A1
(43) Pub. Date: Apr. 19, 2012

(54) Nano- and Micro- Encapsulation of Biomaterials Into Particles and Capsules by Varying Precipitation Conditions

(76) Inventors: Alexander Grinberg, San Francisco, CA (US); Andre Skirtach, Potsdam (DE); Dmitry V. Volodkin, Potsdam (DE); Helmuth Möhwald, Potsdam (DE); Stephan Schmidt, Potsdam (DE); Regine von Klitzing, Berlin (DE)

(21) Appl. No.: 13/272,874

(22) Filed: Oct. 13, 2011

Related U.S. Application Data
(60) Provisional application No. 61/392,476, filed on Oct. 13, 2010.

(51) Int. Cl. A61K 9/16; C07K 14/62; C07K 1/00

(52) U.S. Cl. 427/2.14; 530/402; 530/303

(57) ABSTRACT

This invention describes novel methods for fabricating nano/micro particles and capsules through template decomposition which incorporates the to-be-encapsulated molecules which are precipitated in pores of particles or in solution, i.e. below their isoelectric point, drying or by solvent adjustment methods. The encapsulation process can be followed by a deposition or adsorption of a protective shell that regulates release of the encapsulated material. The encapsulation, inclusion, manipulation, and release of various materials and bio-materials is to be conducted by delivery vehicles which are particles and capsules with sizes in the range of nanometers and micrometers. They can possess multicompartment and anisotropic geometries and can carry one or several types of various molecules. This invention can potentially be used for controlled delivery, manipulation, and release in a variety of applications requiring delivery vehicles such as cell cultures, in-vivo, subcutaneous incorporation, injection, spray-inhalation and planar surfaces, films, and stents.

### CROSS REFERENCE TO RELATED APPLICATIONS

This application claims the priority of U.S. Provisional Application No. 61/392,476 filed Oct. 13, 2010 and entitled, NANO- AND MICRO-ENCAPSULATION OF BIOMATERIALS INTO PARTICLES AND CAPSULES BY VARYING PRECIPITATION CONDITIONS.

### BRIEF DESCRIPTION OF THE DRAWINGS

FIG. 1. Schematic illustrating fabrication of protein microparticles by templating from CaCO₃ micro- and nano-cores. The steps involve loading of CaCO₃ particles or templates with protein by isoelectric precipitation (a-b), removal of the template (b-c) and shrinkage of the porous protein matrix to a compact sphere (c-d). Stability regions of proteins and CaCO₃ templates are shown in the corresponding pH range.

FIG. 2. Scanning electron microscopy images of (a) CaCO₃ template (a broken particles is shown depicting the interior structure), (b) transmission electron microscopy of a protein microsphere and scanning electron microscopy image of a protein sphere, (d) magnified part of (b).

FIG. 3. Adsorption isotherm of protein microspheres. Integral fluorescence is plotted versus initial protein/CaCO₃ weight ratio used for protein loading. (b,c)—confocal scanning laser microscope images at an initial protein/CaCO₃ weight ratio of 15%, transmission and fluorescence mode, respectively.

FIG. 4. a) Diameter d of insulin microspheres and b) protein density 1 in the microspheres as a function of initial protein/CaCO₃ weight ratio used for insulin loading. The diameter of bare CaCO₃ cores (5.5 μm) is presented at zero protein/CaCO₃ weight ratio.

### BACKGROUND OF THE INVENTION

A progressive increase in the number of proteins used as therapeutic agents is driven by the high biological activity and specificity of proteins and also advances in biotechnology, which offers new proteins with tailored therapeutic properties.^{[1]} The use of nano- and microcarriers with proteins is a main strategy for site-specific and prolonged drug delivery. A major challenge in protein drug delivery is the formation of protein particles with well-defined characteristics: size, morphology, composition, and density. These characteristics are critically important to achieve high bioavailability with a particular administration route. Conventional methods to produce protein nano- and microparticles include crystallization,^{[2]} spray- and freeze-drying,^{[3]} and incorporation in polymeric matrices or liposomes.^{[4]} These methods, however, often present significant obstacles for control over particle morphology and size, protein stability due to utilization of organic solvents, and exposure to high temperatures or the gas-water interface. Unforeseen negative impacts of the additives/excipients that are generally used in these methods might also arise. Beyond that, monodispersity is often a key parameter to achieve high systemic bioavailability and welldefined release profiles. Thus, the development of new methods to formulate monodisperse additive-free protein particles is an important challenge.

Nanotechnology is making substantial inputs into the field of material development for drug delivery. Herein we present a new method to fabricate pure micrometer-sized insulin microspheres by templating onto porous pH-decomposable CaCO₃ microcores. Insulin is a glucose-regulating hormone that is used daily by patients suffering from diabetes; we use this important therapeutic protein as a model protein. Insulin particles are formulated by a one-step procedure in aqueous solution without additives or organic solvents. The microspheres are then characterized by optical and electron microscopy to reveal their structure and the mechanism of formation.

Templating by porous sacrificial microparticles composed of calcium carbonate has been introduced as a novel strategy to fabricate polymeric-matrix-type microcapsules at gentle template decomposition conditions (EDTA or acidic pH) using the layer-by-layer approach.^{[5]} The nontoxic nature of these uniform and relatively monodisperse templates, high loading capacity, low price, easy preparation, and mild decomposition conditions stimulated utilization of the cores for template-assisted synthesis to produce biologically active polymeric capsules,^{[6]} multicompartment^{[7]} and stimuliresponsive capsules,^{[8]} and capsules loaded with materials of a different nature, such as organic solvents, pharmaceuticals, enzymes, DNA, phospholipids, and polysaccharides.^{[6a, 9]} Decomposable cores from porous silica have been used as alternative templates to produce microparticles from protein—polymer complexes.^{[10]}

### BRIEF SUMMARY OF THE INVENTION

The present invention describes a method of encapsulating and embedding biomaterials into drug delivery vehicles. The method covers removable templates, particles, and capsules. This entire invention, unless otherwise noted, is done on the level of micrometers and nanometers.

The materials that may be encapsulated through this method include, but are not limited to bio-molecules including polymers, proteins, peptides, bio-polymers, bio-materials, insulin, DNAs, RNAs, other oligonucleotides, therapeutic agents, cytokines, therapeutic agents, medicine, and various medical and prescription drugs.

One example of a template used in this method of encapsulation is calcium carbonate. The process involves the simultaneous formation of the template in the presence of the to-be-encapsulated materials. This process is accomplished by adding, upon mixing, the precipitate forming components forming the templates. The process takes place under stirring conditions. If necessary, the template can be extracted by adding a chelating agent decomposing the template.

The carriers, when they consist of bio-compatible materials, can be used for delivery as the encapsulated materials are packed in their pores. Adsorption of polymers into the carriers can be used to control release, to facilitate specific binding, or to evade binding.

This invention primarily lies in the lowering of pH during the encapsulation process. Upon lowering the pH of the solution in which particles/capsules are situated (i) the templates are decomposed and (ii) the particles/capsules with the encapsulated materials, initially packed in pores of the templates, are released. Thus, reduction of pH on the one hand acts to dissolve the templates, while on the other hand it promotes formation of insoluble particles and capsules.

For example, at higher pH, such as above pH=8, the porous templates are stable and the to-be-encapsulated materials are soluble. Below pH=7 the templates dissolve, while the to-be-encapsulated materials become insoluble. The materials become insoluble at pH values below their isoelectric point (pI).

This method can be applied to a variety of proteins, peptides, and bio-molecules since the pI of most proteins and other bio-molecules is situated in pH range of 4-6. For example, Interferon alfa-n3 (pI=5.99), Human Serum Albumin (pI=5.67), growth hormone Pegvisomant (pI=5.27), and Alpha-1-proteinase inhibitor (pI=5.37).

This method can also be applied to templates whose pores are loaded with encapsulated materials after the templates are synthesized. In this case, direct adsorption of the to-be-encapsulated materials is conducted with the desired amount of template. The template removed as described by reducing the pH of the solution. This approach can be used for controlling the concentration of encapsulated materials, and therefore the size of delivery vehicles.

The size of the delivery vehicles can be controlled in the range of nanometers to millimeters. Control of the size is achieved by varying i) the initial size of templates and ii) the concentration of encapsulated materials. If necessary, these particles/capsules can directly be used as delivery vehicles or covered by a shell as described below.

### DETAILED DESCRIPTION OF THE INVENTION

We report a new method for fabrication of pure micrometer-sized microspheres. The non-toxic nature of these uniform and relatively monodisperse CaCO₃ templates, high loading capacity, low price, and easy preparation and mild decomposition conditions stimulated utilization of the cores for template-assisted synthesis to produce biologically active polymeric capsules; multicompartment and stimuli-responsive capsules; capsules loaded with material of different nature such as organic solvents, pharmaceuticals, enzymes, DNA, phospholipids and polysaccharides. Other cores can also include silica, polystyrene, etc.

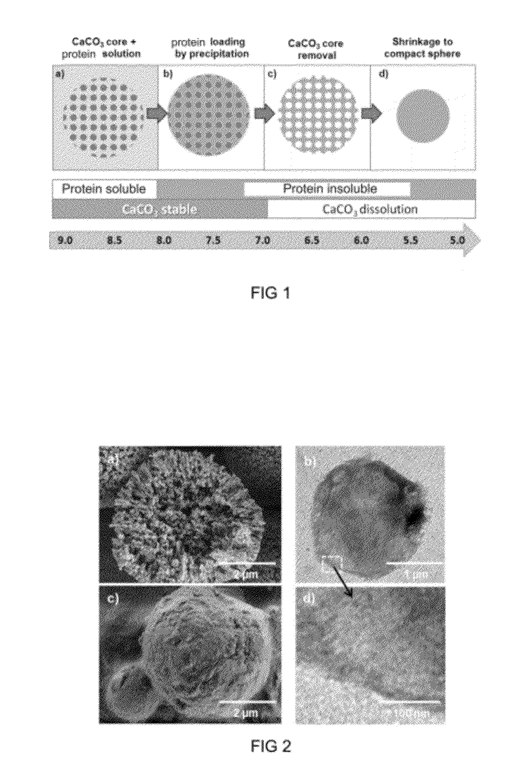

FIG. 1 shows a scheme of microsphere fabrication in one step, i.e. without any additives. Regions of stability of CaCO₃ templates (soluble at acidic pH or in the presence of complexation agents, such as EDTA) and proteins are also shown. If protein solution is titrated with HCl (hydrochloric acid) in the presence of CaCO₃ templates starting from pH 9.5 and ending with pH 5.2 one can observe several intermediate states (FIG. 1). In the vicinity to the isoelectric point the solubility of proteins is dramatically decreased as they become more non-polar in the polar solvent (water) due to the decrease of the net charge of aminoacids. The decrease of the pH below 8.0 induces colloidal instability of proteins that promotes protein flocculation in the pores of CaCO₃ microcores (a-b). Adsorption of protein molecules on the surface of calcium carbonate promotes surface-mediated nucleation which results in the growth of insoluble protein agglomerates in the cores but not in bulk solution. Non-ionic surfactants behave similar aggregating on glass surfaces^{[12]}. During titration at acidic pH the CaCO₃ core is decomposed (b-c) followed by shrinkage of the porous protein matrix to more compact protein microspheres/beads (c-d). The shrinkage is driven by water removal from the pores in the protein matrix, which were created after decomposition of the CaCO₃ template. At the final pH value coinciding with the protein pI (zero net charge of aminoacids), protein-protein interaction is established mostly by hydrophobic interactions which promotes water removal from the pores and particle contraction. Since core decomposition occurs at mild conditions and at strong interprotein interaction it does not induce destruction of the protein matrix (FIG. 1c) as found by analysis of the protein content in the supernatant after core decomposition.

An internal structure of CaCO₃ can be seen in FIG. 2 a. In contrast, protein microspheres are compact beads, FIG. 2 c. Due to shrinkage of the pores in the protein matrix, protein is homogeneously distributed in particles, FIG. 2 b,d.

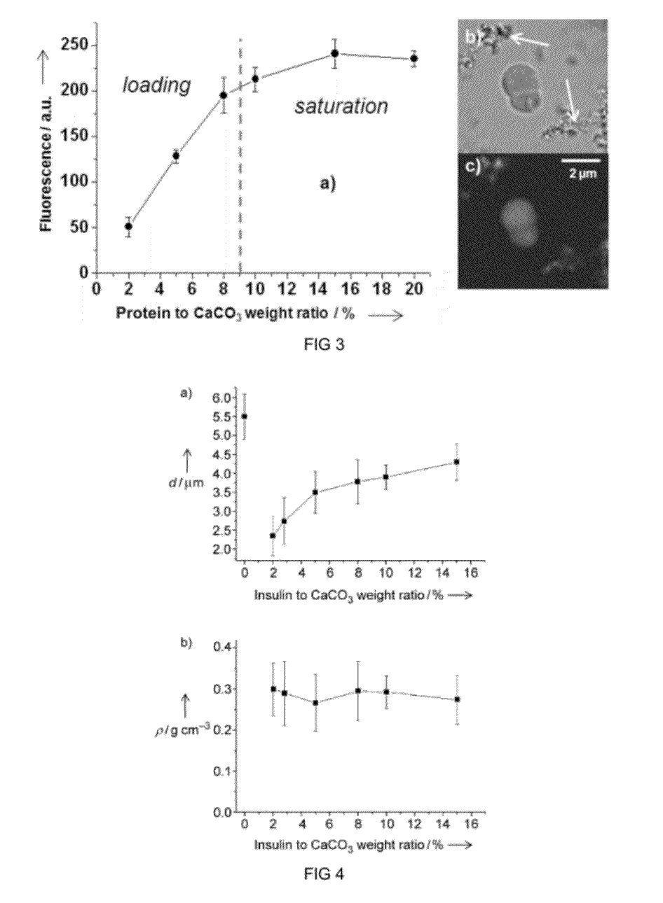

Protein loading in the microspheres has limits. Below the initial protein/CaCO₃ weight ratio 2% the spheres are not formed, most likely because the stability of the protein matrix in the cores is not high enough to compensate the high osmotic pressure created during CaCO₃ core dissolution. A maximum of the loading capacity is reached at a ratio 8-10% (FIG. 3a, depicted by a broken line) that induces an appearance of the protein precipitates in solution together with the microspheres (FIG. 3b,c; bulk precipitates depicted by arrows).

Shrinkage of protein particles takes place after core removal when there is no barrier to prevent collapsing of the porous protein matrix. The contraction extent is considerably increased with decrease of protein loading into the CaCO₃ templates. This can be related to a release of larger amounts of water from the more porous and hydrated protein matrix formed at lower protein loading. The collapsed protein matrix, however, contains a significant amount of water that is independent on initial protein loading into the CaCO₃ cores. The protein density in particles was observed to be around 0.3 g/cm³ for cores loaded with protein at protein/CaCO₃ ratio from 2 to 15%. The above described methods work has been shown for several biomolecules, including insulin which has isoelectric point around 5.3^{[11]}. Precipitation of proteins by pH was reported not to affect the structure of proteins.^{[14]}

FIG. 1 shows microsphere fabrication without any additives and in one step. Regions of stability of CaCO₃ microcores (soluble at acidic pH) and insulin (insoluble in pH range 4.5-7.5; see the Supporting Information) have been identified. If the insulin solution is titrated with hydrochloric acid in the presence of CaCO₃ microcores starting from pH 9.5 and ending with pH 5.2, a few intermediate states are observed (FIG. 1). In the vicinity of the isoelectric point (pI of insulin 5.3^{[11]}), the solubility of insulin is dramatically decreased because it becomes more nonpolar in the polar solvent (water) woing to the decrease of the net charge of aminoacids. The decrease in pH below 8.0 induces a colloidal instability of insulin that promotes protein flocculation in the pores of CaCO₃ microcores (FIG. 1a,b). Adsorption of protein molecules on the surface of calcium carbonate promotes surface-mediated nucleation, which results in the growth of insoluble protein agglomerates in the cores but not in bulk solution. Non-ionic surfactants behave similarly, aggregating on glass surfaces.^{[12]} The CaCO₃ core is slightly negatively charged (ζ potential of about −8 mV)^{[5a]} under these conditions (pH 9.0), which does not prevent protein adsorption (insulin is also negatively charged) on the microcores followed by exclusive precipitation in the pores of the microcores at pH values lower than 9.0. During titration at acidic pH, the CaCO₃ core is decomposed (FIG. 1b,c) followed by shrinkage of the porous insulin matrix to more compact protein microspheres/beads (FIG. 1c,d). The shrinkage is driven by water removal from the pores in the protein matrix; these pores were created after decomposition of the CaCO₃ template. At the final pH value, which coincides with the insulin pI (zero net charge of aminoacids), protein—protein interactions are established mostly by hydrophobic interactions, which promotes water removal from the pores and particle contraction. As core decomposition occurs under mild conditions and with strong interprotein interactions, it does not induce destruction of the protein matrix (FIG. 1c), as found by analysis of the protein content in the supernatant after core decomposition.

A highly developed internal structure of CaCO₃ microcores can be seen in FIG. 2a. In contrast, the insulin microspheres are compact beads (FIG. 2c). Due to shrinkage of the pores in the protein matrix, insulin is homogeneously distributed in the microspheres (FIG. 2b,d), at least on the scale of around 30 nm that corresponds to the pore size of CaCO₃ cores.^{[5b]}

Insulin loading in the microspheres has an upper and a lower limit. Below the initial protein/CaCO₃ weight ratio of 2%, the microspheres are not formed, probably because the stability of the protein matrix in the cores is not high enough to compensate the high osmotic pressure created during CaCO₃ core dissolution. A maximum of the loading capacity is reached at a ratio of 8-10% (FIG. 3a, depicted by a broken line) that induces an appearance of the protein precipitates in solution together with the microspheres (FIG. 3b,c; bulk precipitates depicted by arrows). Taking into account the low content of FITC-labeled insulin molecules (10%) mixed with unlabeled insulin and the low protein density in the microspheres with relatively homogeneous protein distribution, a distance between fluorescein molecules of longer than 10-15 nm can be estimated. Self-quenching, which takes place at interdye distance comparable to the Förster distance (4.2 nm for fluorescein^{[13]}), is thus excluded and the fluorescence intensity is therefore proportional to the dye (that is, protein labeled with the dye) concentration. Shrinkage of insulin microspheres takes place after core removal, when there is no barrier to prevent collapsing of the porous protein matrix. The contraction extent is considerably increased with a decrease of protein loading into the CaCO₃ microcores (FIG. 4a). This effect can be related to a release of larger amounts of water from the more porous and hydrated protein matrix formed at lower protein loadings.

The collapsed protein matrix, however, contains a significant amount of water that is independent on the initial protein loading into the CaCO₃ cores. The protein density in insulin microspheres was found to be around 0.3 g cm⁻³ for cores loaded with protein at protein/CaCO₃ ratios of from 2 to 15% (FIG. 4b). The high water content is not surprising, because insulin molecules are not crystalline and rather amorphous, as shown by small-angle X-ray scattering (SAXS; see Supporting Information).

Amorphous insulin could have some advantages compared to a crystalline phase. Bailey et al. reported that isoelectrical precipitation does not affect the secondary structure of insulin;^{[14]} in general, changes in secondary structure are expected to be less pronounced for the more hydrated amorphous form than for a compact crystalline form. The stability of amorphous insulin towards chemical degradation has been reported to be higher than that of crystalline form.^{[15]} The calculated protein density corroborates well with findings of Bailey et al., who has demonstrated that insulin precipitated in solution at a pH value of about 5 has a density of slightly below 0.3 gcm⁻³ and the content of crystalline insulin is around 5%.^{[14]}

A low protein density is advantageous for pulmonary delivery in deep lungs.^{[16]} Particles prepared in this study have a geometric diameter (d_{g}) from 2 to 4 μm (FIG. 4a) that corresponds to an aerodynamic diameter (dₐ) from 1.1 to 2.2 μm (respirable range^{[17]}), because for spherical particles in water, dₐ is equal to d_{g} multiplied by the square root of the particle density.^{[14]} The finding that the microspheres studied herein have the same protein density as precipitates formed in bulk^{[14]} (FIG. 3b,c) indicates that the shrinking insulin matrix (FIG. 1 c,d) is relatively dynamic but not a frozen structure at the insulin pI.

In conclusion, we show that pure insulin microspheres can be fabricated by protein templating at isoelectric points on decomposable porous microcores from CaCO₃. The main features of the microspheres include uniform size, spherical shape, monodispersity, and no additives or harsh preparation conditions with minimal processing steps. We should stress that the effective method of preparing organic nanoparticles of defined size is not confined to insulin but is of more general applicability. Inspecting FIG. 1, it can be seen that the crucial requirement is an overlap of the template stability and drug solubility along with solubility for a certain parameter (here pH) and otherwise insolubility upon template destruction. CaCO₃ is a suitable decomposable template for many reasons, but also many other proteins or even small drugs fulfill the conditions cited above.

The features of the protein microspheres make the microspheres valuable for protein delivery and show potential to achieve high systemic bioavailability and avoid potential complications owing to the presence of additives. The approach developed herein can be generalized for many other proteins that can be precipitated at conditions under which CaCO₃ microcores are decomposed (that is, acidic pH or the presence of EDTA).

Experimental Section FITC-labeled and unlabeled insulin from bovine pancreas with 0.5% zinc content of was purchased from Sigma (Germany). CaCO₃ microtemplates were prepared according to the procedure described previously,^{[5b]} average particle diameter (5.5±0.6) μm. CaCO₃ particles (10 mg) were dispersed in insulin solution (15 mL) with the pH value adjusted to 9.5. The insulin content was chosen to obtain a protein/CaCO₃ mass ratio from 2 to 20%. Stock insulin contains 10% (w/w) of insulin-FITC. The suspension was slowly titrated with 0.1 m HCl until pH 5.2, followed by dialysis for one day (Float-A-Lyser G2 dialysis tubes, cut-off 0.5-1 kDa, Spectra/Por, USA) against water (2 L) with the pH value adjusted to 5.2. The microspheres were stored at 4° C. as a suspension or lyophilized. All experiments were carried out at room temperature.

The relative content of insulin in the microspheres was calculated using the integral fluorescence from insulin microspheres as a function of initial protein/CaCO₃ weight ratio. The protein density was calculated taking into account an average size, mass, and porosity of CaCO₃ particles^{[5b]} and also the adsorption isotherm (FIG. 3a). 30-40 particles were treated to determine the average cumulative fluorescence (FIG. 3a) and the microsphere diameter and protein density (FIG. 4). For details of CLSM, TEM, SAXS, and insulin titration experiments, see the Supporting Information.

### Extensions

An extension of this method of encapsulation can be used to simultaneously embed several molecules. Embedding is conducted by admixing the to-be-encapsulated materials, molecules, with the template-forming materials while stirring. This is applicable to molecules with different or similar pI. In the case that the molecules have similar pI, encapsulation of two molecules at the same time occurs. In the case where the molecules have different pI, they will precipitate at different times. The molecules with higher pI will precipitate before those with a lower pI. This results in molecules with higher pI forming the outer layers of the end product.

This method can further be extended to create multicompartment particles/capsules. This can be achieved by two or more outer compartments being synthesized by the same process as the core formation (for example direct precipitation upon formation or direct adsorption from a solution or buffer onto or over the template with embedded molecules) as described above. In this case, various molecules can be placed in the different compartments. In the event the template of the end product needs to be removed, the particles/capsules can be formed whose layers are comprised of the encapsulated materials in a desired sequence.

A third extension of this method utilizes anisotropic particles/capsules. Anisotropic particles/capsules can be obtained either from anisotropic templates, which can be synthesized by drastically enhancing the precipitation conditions, or from packing the preformed templates with or without encapsulated materials into a substrate. The substrate can be made as a porous support or a soft, for example gel-like, film. The obtained constructs can be removed from the support or films through either physical when removing supports (for example deformation of the support, application of temperature, etc) or chemical means when removing films (for example adding a solution with acidic pH, weakening the attachment between capsules/particles and the template/film, etc). This process can be further utilized in conjunction with anisotropic, multicompartment particles/capsules.

This method can also be applied to amphiphilic molecules and block co-polymers. The inner core is formed from the molecules with the highest pI. This process results in the formation of micelle-like structured delivery vehicles.

This method can also encapsulate cytokines. These are included within the interior of the particles/capsules and become available for cell signaling upon subsequent release. Methods for release are described below.

A final unique feature of this method applies to forming particles/capsules on planar surfaces, films, and stents. Deposition of porous carbonate is achieved in the first step. Following this, all steps described above can be performed.

### Coatings

When necessary, the above drug delivery vehicles can be coated by polymers, gel-like polymers, antibodies, sol-gel coatings, oil based coatings, hydrophilic polymers, hydrophobic polymers, block co-polymers, block co-polymers with peg blocks, amphiphilic molecules, nano-composite materials, organic nanoparticles, inorganic nanoparticles, metal nanoparticles, magnetic nanoparticles, peg-containing polymers, lipids, or a combination of these or other materials. This step can be used to further control the permeability, control the release profiles, enhance imaging, inducing specific targeting/binding, or elude specific binding. Release profiles are also dependent on the size of the delivery vehicles.

Polymeric nanocomposite coatings can be made from individual polymers and their combinations, such as poly-L-lysine, polyarginine, poly-glutamic acid, gelatin, polysaccharides, chitosan, dextran, and their derivatives.

Smart biodegradable polymers and nanocomposites can also form the coating. The thickness of the coating and coatings as well as the assembly conditions regulate regulates the release, which can be tuned for specific time intervals. Immediate release can also be achieved through the application of external fields. External fields and stimuli can act as the catalyst releasing the capsule contents in applications requiring a specific release sequence. The hybrid organic-inorganic nanocomposites coatings are comprised of as the organic particles, such as polymers, and inorganic particles (such as noble metals, metal oxides, magnetic particles) to provide the release functionality.

Coverage by coatings is performed through depositing a nanocomposite or hybrid nanocomposite (polymeric—particle or nanoparticle) shell onto templates by adsorption, interfacial adsorption, interfacial complexation, surface induced polymerization and deposition, or a combination. Nanocomposite or hybrid nanocomposite coatings are deposited by adsorption, which can depend on factors such as the concentration of salt and pH-values. If necessary coatings can also be applied via spraying.

After coating, templates from bio-inert (for example, silica), biocompatible (for example, calcium carbonate), or bio-degradable materials templates can be used. These templates help add structural support during delivery.

### REFERENCES

[1] G. Walsh, Nat. Biotechnol. 2006, 24, 769.
[2] a) S. K. Basu, C. P. Govardhan, C. W. Jung, A. L. Margolin, Expert Opin. Biol. Ther. 2004, 4, 301; b) S. Pechenov, B. Shenoy, M. X. Yang, S. K. Basu, A. L. Margolin, J. Controlled Release 2004, 96, 149.
[3] a) W. Wang, Int. J. Pharm. 2000, 203, 1; b) Y. F. Maa, P. A. Nguyen, T. Sweeney, S. J. Shire, C. C. Hsu, Pharm. Res. 1999, 16, 249.
[4] a) M. L. Tan, P. F. M. Choong, C. R. Dass, Peptides 2010, 31, 184; b) S. Stolnik, L. Ilium, S. S. Davis, Adv. Drug Delivery Rev. 1995, 16, 195; c) D. V. Volodkin, N. G. Balabushevitch, G. B. Sukhorukov, N. I. Larionova, STP Pharma Sci. 2003, 13, 163; d) D. V. Volodkin, N. G. Balabushevitch, G. B. Sukhorukov, N. I. Larionova, Biochemistry 2003, 68, 236.
[5] a) D. V. Volodkin, N. I. Larionova, G. B. Sukhorukov, Biomacromolecules 2004, 5, 1962; b) D. V. Volodkin, A. I. Petrov, M. Prevot, G. B. Sukhorukov, Langmuir 2004, 20, 3398; c) G. B. Sukhorukov, D. V. Volodkin, A. M. Gunther, A. I. Petrov, D. B. Shenoy, H. Möhwald, J. Mater. Chem. 2004, 14, 2073.
[6] a) B. G. De Geest, R. E. Vandenbroucke, A. M. Guenther, G. B. Sukhorukov, W. E. Hennink, N. N. Sanders, J. Demeester, S. C. De Smedt, Adv. Mater. 2006, 18, 1005; b) G. B. Sukhorukov, A. L. Rogach, M. Garstka, S. Springer, W. J. Parak, A. Munoz-Javier, O. Kreft, A. G. Skirtach, A. S. Susha, Y. Ramaye, R. Palankar, M. Winterhalter, Small 2007, 3, 944.
[7] O. Kreft, M. Prevot, H. Mohwald, Gleb B. Sukhorukov, Angew. Chem. 2007, 119, 5702; Angew. Chem. Int. Ed. 2007, 46, 5605.
[8] C. Dejugnat, G. B. Sukhorukov, Langmuir 2004, 20, 7265.
[9] a) C. Wang, C. He, Z. Tong, X. Liu, B. Ren, F. Zeng, Int. J. Pharm. 2006, 308, 160; b) C. Deng, W.-F. Dong, T. Adalsteinsson, J. K. Ferri, G. B. Sukhorukov, H. Möhwald, Soft Matter 2007, 3, 1293; c) J. Li, Z. Y. Jiang, H. Wu, L. Zhang, L. H. Long, Y. J. Jiang, Soft Matter 2010, 6, 542; d) T. Borodina, E. Markvicheva, S. Kunizhev, H. Möhwald, G. B. Sukhorukov, O. Kreft, Macromol. Rapid Commun. 2007, 28, 1894; e) K. Gopal, Z. Lu, M. M. de Villiers, Y. Lvov, J. Phys. Chem. B 2006, 110, 2471.
[10] Y. J. Wang, F. Caruso, Adv. Mater. 2006, 18, 795.
[11] A. Conwayja, L. M. Lewin, Anal. Biochem. 1971, 43, 394.
[12] O. Dietsch, A. Eltekov, H. Bock, K. E. Gubbins, G. H. Findenegg, J. Phys. Chem. C 2007, 111, 16045.
[13] A. Kawski, Photochem. Photobiol. 1983, 38, 487.
[14] M. M. Bailey, E. M. Gorman, E. J. Munson, C. Berkland, Langmuir 2008, 24, 13614.
[15] M. J. Pikal, D. R. Rigsbee, Pharm. Res. 1997, 14, 1379.
[16] D. A. Edwards, J. Hanes, G. Caponetti, J. Hrkach, A. Ben-Jebria, M. L. Eskew, J. Mintzes, D. Deaver, N. Lotan, R. Langer, Science 1997, 276, 1868.
[17] a) E.-S. Khafagy, M. Morishita, Y. Onuki, K. Takayama, Adv. Drug Delivery Rev. 2007, 59, 1521; b) J. Patton, Curr Med Res. Opin 2006, 22, S5.

What is claimed is:

1. The method of synthesizing particles or capsules by lowering pH during particle or capsule fabrication.

2. The method of claim 1 where pH is lowered below the isoelectric point of the to-be-encapsulated materials.

3. The method of claim 1 performed in the presence of the to-be-encapsulated materials and the porous template.

4. The method of claim 3 where the template is simultaneously extracted while forming the particles or capsules.

5. The method of claim 3 where the lowering of pH causes the to-be-encapsulated materials to enter the porous template.

6. The method of claim 3 where the lowering of pH aids in causing the to-be-encapsulated materials to enter the porous template.
